(19) United States
(12) Patent Application Publication
(10) Pub. No.: US 2005/0137209 A1
(43) Pub. Date: Jun. 23, 2005

(54) Treatment of neurologic disorders with inhibitors of 11beta-HSD1

(75) Inventors: Donna Oksenberg, Palo Alto, CA (US); Mehrdad Shamloo, Foster City, CA (US); Roman Urfer, Belmont, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: AGY Therapeutics, Inc.

(21) Appl. No.: 11/016,387

(22) Filed: Dec. 17, 2004

Related U.S. Application Data
(60) Provisional application No. 60/577,835, filed on Jun. 7, 2004.
(60) Provisional application No. 60/531,182, filed on Dec. 18, 2003.

(51) Int. Cl. A61K 31/496

(52) U.S. Cl. 514/254.02

(57) ABSTRACT

Methods and compositions for the treatment of neurologic disorders involving neuronal death, including but not limited to focal or global ischemia of the brain and central nervous system. In vivo inhibition of 11 beta hydroxysteroid dehydrogenase 1 (HSD1) is shown to be neuroprotective in these conditions. HSD1 inhibitors are administered alone or in combination with additional agents for prophylaxis or therapy.

Neurodegenerative diseases are characterized by the dysfunction and death of neurons, leading to the loss of neurologic functions mediated by the brain, spinal cord and the peripheral nervous system. These disorders have a major impact on society. For example, approximately 4 to 5 million Americans are afflicted with the chronic neurodegenerative disease known as Alzheimer's disease. Other examples of chronic neurodegenerative diseases include diabetic peripheral neuropathy, multiple sclerosis, amyotrophic lateral sclerosis, Huntington's disease and Parkinson's disease. Normal brain aging is also associated with loss of normal neuronal function and may entail the depletion of certain neurons.

Though the mechanisms responsible for the dysfunction and death of neurons in neurodegenerative disorders are not well understood, a common theme is that loss of neurons results in both the loss of normal functions and the onset of adverse behavioral symptoms. Therapeutic agents that have been developed to retard loss of neuronal activity and survival have been largely ineffective. Some have toxic side effects that limit their usefulness. Other promising therapies, such as neurotrophic factors, are prevented from reaching their target site because of their inability to cross the blood-brain barrier.

Stroke is the third ranking cause of death in the United States, and accounts for half of neurology inpatients. Depending on the area of the brain that is damaged, a stroke can cause coma, paralysis, speech problems and dementia. The five major causes of cerebral infarction are vascular thrombosis, cerebral embolism, hypotension, hypertensive hemorrhage, and anoxia/hypoxia.

The brain requires glucose and oxygen to maintain neuronal metabolism and function. Hypoxia refers to inadequate delivery of oxygen to the brain, and ischemia results from insufficient cerebral blood flow. The consequences of cerebral ischemia depend on the degree and duration of reduced cerebral blood flow. Neurons can tolerate ischemia for 30-60 minutes, but perfusion must be reestablished before 3-6 hours of ischemia have elapsed. Neuronal damage can be less severe and reversible if flow is restored within a few hours, providing a window of opportunity for intervention.

If flow is not reestablished to the ischemic area, a series of metabolic processes ensue. The neurons become depleted of ATP and switch over to anaerobic glycolysis (Yamane et al. (2000) J Neurosci Methods 103(2):163-71). Lactate accumulates and the intracellular pH decreases. Without an adequate supply of ATP, membrane ion pumps fail. There is an influx of sodium, water, and calcium into the cell. The excess calcium is detrimental to cell function and contributes to membrane lysis. Cessation of mitochondrial function signals neuronal death (Reichert et al. (2001) J Neurosci. 21(17):6608-16). The astrocytes and oligodendroglia are slightly more resistant to ischemia, but their demise follows shortly if blood flow is not restored (Sochocka et al. (1994) Brain Res 638(1-2): 21-8).

Evidence is also emerging in support of the possibility that acute inflammatory reactions to brain ischemia are causally related to brain damage. The inflammatory condition consists of cells (neutrophils at the onset and later monocytes) and mediators (cytokines, chemokines, others). Upregulation of proinflammatory cytokines, chemokines and endothelial-leukocyte adhesion molecules in the brain follow soon after an ischemic insult and at a time when the cellular component is evolving. The significance of the inflammatory response to brain ischemia is not fully understood (Feuerstein et al. (1997) Ann N Y Acad Sci 825:179-93).

In animal models of middle cerebral artery occlusion, it has been found that an ischemic penumbra surrounds a focus of dense cerebral ischemia. The ischemic penumbra is the region where cerebral blood flow reduction has exceeded the threshold for failure of electrical function but not that for membrane failure. The ischemic core region enlarges when adjacent, formerly penumbral, areas undergo irreversible deterioration during the initial hours of vascular occlusion. At the same time, the residual penumbra becomes restricted to the periphery of the ischemic territory, and its fate may depend critically upon early therapeutic intervention.

Electrophysiological measurements show penumbral cell depolarizations, associated with an increased metabolic workload, which induce episodes of tissue hypoxia. The frequency of their occurrence correlates with the final volume of ischemic injury. Therefore, penumbral depolarizations have been thought to be important in the pathogenesis of ischemic brain injury. Periinfarct direct current deflections can be suppressed by NMDA Receptor and non-NMDA Receptor antagonists, resulting in a significant reduction of infarct size (Back (1998) Cell Mol Neurobiol. 18(6):621-38). The histopathological sequelae within the penumbra consist of various degrees of scattered neuronal injury, also termed “incomplete infarction.” (Lassen (1984) Stroke 15(4):755-8) The reduction of neuronal density at the infarct border is a flow- and time-dependent event, which is affected by the activity of astrocytes and glial cells. Thus, the penumbra is a spatially dynamic brain region of limited viability, which is characterized by complex pathophysiological changes in response to local ischemic injury.

The treatment of stroke includes preventive therapies, such as antihypertensive and antiplatelet drugs, which control and reduce blood pressure and thus reduce the likelihood of stroke. Also, the development of thrombolytic drugs such as t-PA (tissue plasminogen activator) has provided a significant advance in the treatment of ischemic stroke victims, although to be effective it is necessary to begin treatment very early, within about three hours after the onset of symptoms. These drugs dissolve blood vessel clots which block blood flow to the brain and which are the cause of approximately 80% of strokes (see for reviews, Kent et al. (2001) Stroke 32(10):2318-27; and Albers (2001) Neurology 57(5 Suppl 2):S77-81). However, these drugs can also present the side effect of increased risk of bleeding, and t-PA has recently been shown to have direct neurotoxic effects (Flavin et al. (2000) Glia 29(4):347-54). Various neuroprotectors, such as calcium channel antagonists, can sometimes stop damage to the brain as a result of ischemic insult (Horn et al. (2001) Stroke 32(2):570-6). The window of treatment for these drugs is typically broader than that for the clot dissolvers and they do not increase the risk of bleeding.

Development of methods of treatment for stroke and neurodegenerative conditions is of great clinical interest.

### SUMMARY OF THE INVENTION

The present invention relates to methods and compositions for the treatment of neurologic disorders associated with neuronal death, including but not limited to focal or global ischemia of the brain and central nervous system, traumatic brain injury and Parkinson's disease. Specifically, in vivo inhibition of 11β-hydroxysteroid dehydrogenase 1 (HSD1) is shown to be neuroprotective. HSD1 inhibitors are administered alone or in combination with additional agents for prophylaxis or therapy.

In one embodiment of the invention, the neuroprotective agent is a selective inhibitor of HSD1, and substantially lacks inhibitory activity against HSD2. The neuroprotective agent may be provided as a pharmaceutical composition suitable for in vivo administration to the brain or central nervous system, comprising a pharmaceutically acceptable excipient, and in a dose effective for the prevention or treatment of neurodegeneration in vivo. A packaged kit for clinical use may include a pharmaceutical formulation of an HSD1 inhibitor, a container housing the pharmaceutical formulation during storage and prior to administration, and instructions, e.g., written instructions on a package insert or label, for carrying out drug administration in a manner effective to treat or prevent neurologic disorders involving neuronal death.

In another embodiment, methods are provided for treating or preventing neurologic disorders involving neuronal death in a subject, the method comprising administering a pharmaceutically effective amount of an HSD1 inhibitor, preferably a selective HSD1 inhibitor, to the subject. Administration may be systemic or localized to the brain.

The invention also provides methods for the identification of compounds that selectively inhibit HSD-1 and are therapeutically useful in the treatment of neurologic disorders involving neuronal death.

### BRIEF DESCRIPTION OF THE FIGURES

FIG. 1 demonstrates the experimental design for the in vivo efficacy study (top) as well as % brain infarction following MCAO with and without treatment with selective HSD1 inhibitor.

FIG. 2 illustrates the neuroprotective effects of CBX in animals subjected to MCAO.

### DETAILED DESCRIPTION OF THE EMBODIMENTS

Methods are provided for treating or preventing neurologic disorders involving neuronal death in a subject, including but not limited to focal or global ischemia of the brain and central nervous system, traumatic brain injury and Parkinson's disease, and the method comprising administering a pharmaceutically effective amount of an HSD1 inhibitor to the subject. Administration may be systemic or localized to the brain.

In some embodiments of the invention, the HSD1 inhibitor is selective for HSD1. Selective inhibitors may be preferred in order to minimize side-effects of drug administration. 11β-HSD2 action, which converts cortisol to cortisone, prevents the activation of the mineralocorticoid receptor by cortisol and protects it from glucocorticoid occupation. 11β-HSD2 is expressed in mineralocorticoid responsive tissues such as the kidney and in the placenta where it protects the fetus from the high level of maternal serum cortisol. For example, a deficiency of 11β-HSD2 may lead to severe hypermineralocorticoid-like changes such as hypertension, suppressed rennin and aldosterone levels, water and sodium retention and hypokalaemia (see Stewart et al. (1988) J. Clin. Invest. 82:340-349; and Stewart (1990) Clin. Sc. 78:49-54). In addition, studies in hypertensive patients (Walker B R et al, 1991, J. Endocrinol, Vol 129, p. 282s; Walker B R et al, 1991, J. Hypertens. Vol 9, p1082-1083) have produced evidence of a slower than normal clearance of cortisol and an increase in vascular sensitivity to cortisol that may be due to altered target-organ 11β-HSD2 activity.

### Disease Conditions

“Neurologic disorder” is defined here and in the claims as a disorder in which loss of neurons occurs either in the peripheral nervous system or in the central nervous system. Examples of neurologic disorders include: chronic diseases such as Parkinson's disease and Huntington's chorea, and acute disorders including: stroke, traumatic brain injury, peripheral nerve damage, spinal cord injury, anoxia, and hypoxia. For example, neuronal death may be a sequelae to exposure to hypoxia, or ischemia.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardic arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). These focal ischemia models are in contrast to global ischemia models where blood flow to the entire brain is blocked for a period of time prior to reperfusion. Certain regions of the brain are particularly sensitive to this type of ischemic insult. The precise region of the brain that is directly affected is dictated by the location of the blockage and duration of ischemia prior to reperfusion. One model for focal cerebral ischemia uses middle cerebral artery occlusion (MCAO) in rats. Studies in normotensive rats can produce a standardized and reproducible infarction. MCAO in the rat mimics the increase in plasma catecholamines, electrocardiographic changes, sympathetic nerve discharge, and myocytolysis seen in the human patient population.

The methods of the invention are also useful for treatment of injuries to the central nervous system that are caused by mechanical forces, such as a blow to the head or spine, and which, in the absence of treatment, result in neuronal death. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

Diagnosis. Various methods are available for the diagnosis of stroke. A focused, prompt, and precise diagnosis is particularly helpful, because the window for preventing neuronal death is relatively narrow, preferably less than as soon as possible after the onset of the symptoms. The administration can be initiated within the first 48 hours of the onset of the symptoms, preferably within the first 24 hours of the onset of the symptoms, more preferably within about 12 hours to about 15 hours of the onset of the symptoms, more preferably within the first 6 hours, even more preferably within the first 3 hours of the onset of the symptoms, and most preferably within about 5 min. to about 3 hours of the onset of the symptoms. Thus, the initial administration can be at about 15 min., 0.5 h, 1 h, 1.5 h, 2 h, 3 h, and so on after the onset of the symptoms.

The abrupt presentation of acute ischemic stroke results from the abrupt interruption of blood flow to a part of the brain. Most commonly this is from embolic or thrombotic arterial vascular occlusion, which may be visualized angiographically if symptoms are severe enough to warrant acute angiography. Other vascular events that can result in stroke syndromes include lacunar strokes, arteritis, arterial dissections, and cortical venous occlusions. Intraparenchymal intracranial hemorrhage from a variety of causes including spontaneous or hypertensive hemorrhages, vascular malformations, or aneurysmal origin are frequently encountered clinically and figure prominently in the initial stroke differential diagnosis. Other tools for diagnosis include magnetic resonance imaging (MRI), magnetic resonance angiography (MRA), diffusion-weighted imaging (DWI), and perfusion-weighted imaging (PWI) to investigate patients thought to have anterior circulation stroke.

Most strokes present as a deficit or loss of function. Uncommonly, movement disorders will present from a focal lesion such as ischemic stroke or hemorrhage. Acute hemiballismus, or unilateral dyskinesis, often result from acute vascular lesions in the subthalamic nucleus or connections. The movements may vary from wild flinging movements to mild uncontrollable unilateral movements. The key to diagnosis is the abrupt onset of symptoms and risk factors for cerebrovascular disease. Confusional states, agitation, and delirium have all been reported as a consequence of focal neurologic injury; structures involving the limbic cortex of the temporal lobes and the orbitofrontal regions are commonly involved. Sensory complaints of either unusual sensations or loss of sensation are common in parietal and thalamic strokes. At times the sensory manifestation of a stroke may take on the characteristics of another clinical condition. Chest pain and limb pain that mimicked that of myocardial infarction were reported in a small series of patients; most had thalamic strokes but one had a lateral medullary infarct.

The American Heart Association (AHA) has issued practice guidelines for the use of imaging tests in stroke patients (Culebras et al. (1997) Stroke 28:1480-1497). CT of the head without contrast enhancement is recommended by the AHA for initial brain imaging. If those study results are negative, a follow-up scan is recommended 2 to 7 days after stroke onset.

Parkinson's disease. Parkinson's Disease is an idiopathic, slowly progressive, degenerative CNS disorder characterized by slow and decreased movement, muscular rigidity, resting tremor, and postural instability. It affects about 1% of those >=65 years old and 0.4% of those >40 years old. The mean age of onset is about 57 yr. It may begin in childhood or adolescence (juvenile parkinsonism).

In primary Parkinson's disease, the pigmented neurons of the substantia nigra, locus caeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Coadministration of an anticholinergic drug (eg, benztropine 0.2 to 2 mg per-oral administration (po) tid (trice daily administration) or amantadine (100 mg po bid (twice daily administration) may ameliorate the resulting symptoms. Less common causes include carbon monoxide or manganese poisoning, hydrocephalus, structural lesions (tumors, infarcts affecting the midbrain or basal ganglia), subdural hematoma, and degenerative disorders, including striatonigral degeneration and multiple systems atrophy. N-MPTP (n-methyl-1,2,3,4-tetrahydropyridine) can cause severe, sudden, and irreversible parkinsonism in intravenous (IV) drug abusers.

Early signs, including infrequent blinking and lack of facial expression, decreased movement, impaired postural reflexes, and the characteristic gait abnormality, suggest the disease. Tremor occurs initially in about 70% of patients but often becomes less prominent as the disease progresses. Although rigidity is occasionally minimal or lacking, tremor without the above features suggests an alternate diagnosis or the need for a later reevaluation, because additional signs will develop if the patient has Parkinson's disease. Causes of the disease may be discerned from the history.

Conventional drug therapy includes Levodopa, the metabolic precursor of dopamine, which crosses the blood-brain barrier into the basal ganglia where it is decarboxylated to form dopamine, replacing the missing neurotransmitter. Coadministration of the peripheral decarboxylase inhibitor carbidopa lowers dosage requirements by preventing levodopa catabolism, thus decreasing side effects (nausea, palpitations, flushing) and allowing more efficient delivery of levodopa to the brain. Most patients require 400 to 1000 mg/day of levodopa in divided doses qid (four times daily) 2 to 5 hours with at least 100 mg/day of carbidopa to minimize peripheral side effects. Some patients may require up to 2000 mg/day of levodopa with 200 mg of carbidopa. After 2 to 5 years of treatment, >50% of patients begin to experience fluctuations in their response to levodopa (on-off effect). The duration of improvement after each dose of drug shortens, and superimposition of dyskinetic movements results in swings from intense akinesia to uncontrollable hyperactivity. Traditionally, such swings are managed by keeping individual doses of levodopa as low as possible and using dosing intervals as short as 1 to 2 hours. Dopamine-agonist drugs, controlled-release levodopa/carbidopa, or selegiline (see below) may be useful adjuncts. Other side effects of levodopa include orthostatic hypotension, nightmares, hallucinations, and, occasionally, toxic delirium. Hallucinations and delirium are most common in elderly, demented patients.

Amantadine 100 to 300 mg/day po is useful in treating early, mild parkinsonism for 50% of patients and in augmenting the effects of levodopa later in the disease. Its mechanism of action is uncertain; it may act by augmenting dopaminergic activity, anticholinergic effects, or both. Amantadine often loses its effectiveness after a period of months when used alone. Side effects include lower extremity edema, livedo reticularis, and confusion.

Bromocriptine and pergolide are ergot alkaloids that directly activate dopamine receptors in the basal ganglia. Bromocriptine 5 to 60 mg/day or pergolide 0.1 to 5.0 mg/day po is useful at all stages of the disease, particularly in later stages when response to levodopa diminishes or on-off effects are prominent. Use is often limited by a high incidence of adverse effects, including nausea, orthostatic hypotension, confusion, delirium, and psychosis. Bromocriptine or pergolide can rarely be used as the sole antiparkinsonian drug. New dopamine agonists that are more specific for the D2 receptor include pramipexole and ropinirole.

Selegiline, a monoamine oxidase type B (MAO-B) inhibitor, inhibits one of the two major enzymes that breaks down dopamine in the brain, thereby prolonging the action of individual doses of levodopa. At doses of 5 to 10 mg/day po, it does not cause hypertensive crisis, common with nonselective MAO inhibitors, which block the A and B isoenzymes. In some patients with mild on-off problems, selegiline helps diminish the end-of-dose wearing off of levodopa's effect. Although virtually free of side effects, selegiline can potentiate the dyskinesias, mental adverse effects, and nausea produced by levodopa, and the dose of levodopa may have to be reduced.

Anticholinergic drugs are used alone in the early stages of treatment and later to supplement levodopa. Commonly used anticholinergics include benztropine 0.5 to 2 mg po tid and trihexyphenidyl 2 to 5 mg po tid. Antihistamines with anticholinergic action (eg, diphenhydramine 25 to 200 mg/day po and orphenadrine 50 to 200 mg/day po) are useful for treating tremor. Anticholinergic tricyclic antidepressants (eg, amitriptyline 10 to 150 mg po at bedtime) often are useful as adjuvants to levodopa, as well as in treating depression. Initially, the dose should be small, and then increased as tolerated. Catechol O-methyltransferase (COMT) inhibitors, such as tolcapone and entacapone, inhibit the breakdown of dopamine and therefore appear to be useful as adjuncts to levodopa. Propranolol 10 mg bid to 40 mg po qid occasionally helps when parkinsonian tremor is accentuated rather than quieted by activity or intention.

Traumatic Brain Injury. Head injury causes more deaths and disability than any other neurologic condition before age 50 and occurs in >70% of accidents, which are the leading cause of death in men and boys <35 years old. Mortality from severe injury approaches 50% and is only modestly reduced by current treatment. Damage may result from skull penetration or from rapid brain acceleration or deceleration, which injures tissue at the point of impact, at its opposite pole (contrecoup), or diffusely within the frontal and temporal lobes. Nerve tissue, blood vessels, and meninges can be sheared, torn, or ruptured, resulting in neural disruption, intracerebral or extracerebral ischemia or hemorrhage, and cerebral edema. Hemorrhage and edema act as expanding intracranial lesions, causing focal neurologic deficits or increased intracranial swelling and pressure, which can lead to fatal herniation of brain tissue through the tentorium or foramen magnum. Skull fractures may lacerate meningeal arteries or large venous sinuses, producing epidural or subdural hematoma. Fractures, especially at the skull base, can also lacerate the meninges, causing CSF to leak through the nose (rhinorrhea) or ear (otorrhea) or bacteria or air to enter the cranial vault. Infectious organisms may reach the meninges via cryptic fractures, especially if they involve the paranasal sinuses.

Concussion is characterized by transient posttraumatic loss of awareness or memory, lasting from seconds to minutes, without causing gross structural lesions in the brain and without leaving serious neurologic residua. Patients with concussion rarely are deeply unresponsive. Pupillary reactions and other signs of brain stem function are intact; extensor plantar responses may be present briefly but neither hemiplegia nor decerebrate postural responses to noxious stimulation appear. Lumbar puncture is generally contraindicated in cases of head trauma unless meningitis is suspected and should be performed only after appropriate x-rays or imaging studies. Postconcussion syndrome commonly follows a mild head injury, more often than a severe one. It includes headache, dizziness, difficulty in concentration, variable amnesia, depression, apathy, and anxiety. Considerable disability can result. Studies suggest that even mild trauma can cause neuronal damage.

Cerebral contusions and lacerations are more severe injuries. Depending on severity, they are often accompanied by severe surface wounds and by basilar skull fractures or depression fractures. Hemiplegia or other focal signs of cortical dysfunction are common. More severe injuries may cause severe brain edema, producing decorticate rigidity (arms flexed and adducted, legs and often trunk extended) or decerebrate rigidity (jaws clenched, neck retracted, all limbs extended). Coma, hemiplegia, unilaterally or bilaterally dilated and unreactive pupils, and respiratory irregularity may result from initial trauma or internal brain herniation and require immediate therapy. Increased intracranial pressure, producing compression or distortion of the brain stem, sometimes causes BP to rise and pulse and respiration to slow (Cushing's phenomenon). Brain scans may reveal bloody CSF; lumbar puncture is usually contraindicated.

Nonpenetrating trauma is more likely to affect the cerebral hemispheres and underlying diencephalon, which are larger and generally more exposed, than the brain stem. Thus, signs of primary brain stem injury (coma, irregular breathing, fixation of the pupils to light, loss of oculovestibular reflexes, diffuse motor flaccidity) almost always imply severe injury and poor prognosis.

Thoracic damage often accompanies severe head injuries, producing pulmonary edema (some of which is neurogenic), hypoxia, and unstable circulation. Injury to the cervical spine can damage the spinal cord, causing fatal respiratory paralysis or permanent quadriplegia. Proper immobilization should be maintained until stability of the cervical spine has been documented by appropriate imaging studies.

Acute subdural hematomas (blood between the dura mater and arachnoid, usually from bleeding of the bridging veins) and intracerebral hematomas are common in severe head injury. Along with severe brain edema, they account for most fatalities. All three conditions can cause transtentorial herniation with deepening coma, widening pulse pressure, pupils in midposition or dilated and fixed, spastic hemiplegia with hyperreflexia, quadrispasticity, decorticate rigidity, or decerebrate rigidity (due to progressive rostral-caudal neurologic deterioration). CT or MRI scans can usually identify operable lesions. Surgical excision of large lesions may be lifesaving, but posttraumatic morbidity is often high.

Epidural hematomas (blood between the skull and dura mater) are caused by arterial bleeding, most commonly from damage to the middle meningeal artery. Symptoms usually develop within hours of the injury and consist of increasing headache, deterioration of consciousness, motor dysfunction, and pupillary changes. A lucid interval of relative neurologic normality often precedes neurologic symptoms. Epidural hematoma is less common than subdural hematoma but is important because prompt evacuation can prevent rapid brain shift and compression, which can cause fatal or permanent neurologic deficits. Temporal fracture lines suggest the diagnosis but may not always be seen on skull x-rays. CT or MRI scans or angiograms should be obtained promptly. If scans are unavailable, burr holes should be drilled promptly to aid diagnosis and allow evacuation of the clot.

Current recommendations for treatment include giving an anticonvulsant for 2 wk; eg, phenytoin may be given as a loading dose of 50 mg/min IV to a total of 1 g followed by 300 to 400 mg/day po or IV. Osmotic diuretics (urea, mannitol, glycerol) given IV reduce brain swelling but should be reserved for deteriorating patients or for preoperative use in patients with hematomas. For those with hematomas, mannitol 12.5 to 25 g is given IV over 15 to 30 min and repeated q 1 to 4 h. It must be used cautiously in patients with heart disease or pulmonary vascular congestion because it induces rapid expansion of vascular volume. Because osmotic diuretics increase renal excretion of water relative to sodium, prolonged use may result in water depletion and hypernatremia. Fluid and electrolyte balance should be monitored. Corticosteroids are contraindicated in head injury.

HSD. 11 β-hydroxysteroid dehydrogenases are enzymes that metabolize glucocorticoids and hence regulate the intracelluler level of steroid available to activate corticosteroid receptors. There are two isoenzymes, 11β HSD type 1 and type 2, which in most tissues and conditions drive the enzyme reaction in opposite directions. 11 β-HSD1 is bidirectional in vitro, but in vivo acts as a NADPH-dependent reductase catalyzing the conversion of inactive cortisone to hormonally active cortisol in humans. The type II isoform only catalyzes the cortisol to cortisone reaction. HSD1 has been detected in a wide range of rat and human tissues, including liver, lung, brain, bone and testis. HSD2 is expressed predominantly in the kidney and placenta. The coding sequences of these genes are only 21% identical.

The human HSD1 sequence is publicly available, for example at Genbank, accession number P28845, and as described by Tannin (1991) J. Biol. Chem. 266 (25), 16653-16658. The human HSD2 sequence is available at Genbank, accession number U26726, as described by Brown et al. (1996) Biochem. J. 313 (Pt 3), 1007-1017.

Preferred inhibitors are selective for HSD1, and are substantially free of HSD2 inhibitory activity. Generally, in the presence of the inhibitor, the enzymatic activity of HSD2 is at least about 90% of the activity in the absence of the compound, more usually at least about 95%, and may be 99% or higher. The IC50 may be used as a measure of the selectivity of the inhibitor, where the IC50 of the inhibitor for the targeted HSD1 protein of interest, i.e. human, mouse, etc., will be less than about 5000 nM, usually less than 500 nM, preferably less than about 250 nM, and may be less than about 100 nM. The IC50 for the compound against HSD2 will generally be greater than about 5000 nM, usually greater than about 10,000 nM.

One of skill in the art can readily assess the selectivity of candidate HSD1 inhibitors. As discussed above, glucocorticoid activity is controlled by intracellular interconversion of active cortisol and inactive cortisone by the 11β-hydroxysteroid dehydrogenases, 11β-HSD1, which catalyzes the reduction of cortisone to cortisol and 11β-HSD2, which converts cortisol to cortisone. Both enzymes have important functional differences such as cofactor specificity, substrate affinity and direction of the reaction. The activity of 11β-HSD1 can be specifically measured by looking at the conversion of cortisone to cortisol. Assessment of 11β-HSD2 activity is based on the conversion of cortisol to cortisone. For example, see Schweizer et al. (2003) Mo. Cell. Endocrin. 212:41-49, herein specifically incorporated by reference.

In such a selectivity assay, the human or murine 11β-HSD1 can be transiently expressed in HEK 293 cells and the lysates can be used as source for the enzyme (see Schweizer et al. (2004) J.B.C. 279 (18): 18415-18424). The human 11β-HSD1 can also be cloned, expressed in E. coli and purified (Hosfield, D. J. et al. (2004) JBC published as Manuscript M411104200). The 11β-HSD2 can also be transiently expressed in HEK 293 cells and the lysates are used as a source for the enzyme (Odermatt et al. (1999) J.B.C. 274 (40): 28762-28770).

Useful assays for this purpose include a scintillation proximity assay for 11β-HSD1 inhibitors (see Barf et al (2002) J. Med Chem, 45(18):3813-3815). Reactions are initiated by addition of human 11β-HSD1 either from cell lysates or the purified enzyme. Following mixing the plates are shaken for 45 minutes at room temperature. The reactions are terminated by addition of a stop solution. Monoclonal anti-cortisol antibody is then added, followed by SPA beads. Appropriate controls are set up in absence of the 11β-HSD1 to obtain the non-specific binding. The amount of [³H]-cortisol bound to the beads is determined in a microplate beta scintillation counter. The IC50 (concentration of the inhibitor that inhibits 50% of the 11β-HSD1) can be determined.

The assessment of 11β-HSD2 activity is based on the conversion of [³H] cortisol to [³H] cortisone in the presence of inhibitor. The enzymatic reaction is performed in presence of NAD and the enzyme and stopped with perchloric acid. Both substrate and product are separated by HPLC and monitored using a flow scintillation counter. Enzyme activity is quantified as the percentage area of the product compared to the total area.

Inhibitors may be provided as a “pharmaceutically acceptable salt”, by which is intended a salt that is pharmaceutically acceptable and that possesses the desired pharmacological activity of the parent compound. Such salts, for example, include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;
(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “optional” or “optionally” means that the subsequently described event or circumstance may or may not occur, and that the description includes instances where the event or circumstance occurs and instances where it does not. For example, the phrase “optionally another drug” means that the patient may or may not be given a drug other than the selective HSD1 inhibitor. “Another drug” as used herein is meant any chemical material or compound suitable for administration to a mammalian, preferably human, which induces a desired local or systemic effect. In general, this includes: anorexics; anti-infectives such as antibiotics and antiviral agents, including many penicillins and cephalosporins; analgesics and analgesic combinations; antiarrhythmics; antiarthritics; antiasthmatic agents; anticholinergics; anticonvulsants; antidiabetic agents; antidiarrheals; antihelminthics; antihistamines; antiinflammatory agents; antimigraine preparations; antinauseants; antineoplastics; antiparkinsonism drugs; antipruritics; antipsychotics; antipyretics; antisense agents; antispasmodics; cardiovascular preparations including calcium channel blockers and beta-blockers such as pindolol; antihypertensives; central nervous system stimulants; cough and cold preparations, including decongestants; diuretics; gastrointestinal drugs, including H₂-receptor antagonists; sympathomimetics; hormones such as estradiol and other steroids, including corticosteroids; hypnotics; immunosuppressives; muscle relaxants; parasympatholytics; psychostimulants; sedatives; tranquilizers; and vasodilators.

### Methods of Treatment

In the methods of the invention, selective inhibitors of HSD1 are administered in vivo to a patient that have suffered a neurologic disorder associated with neuronal death, as well as prophylactically treating individuals at risk for a neurologic disorder associated with neuronal death. In general, such methods involve administering to an individual that has suffered or is at risk for such a neurologic disorder, a selective inhibitor of HSD1 in an amount effective to decrease the expression or activity of HSD1 in the affected tissue, i.e. central nervous system or brain. The neurological injury being treated can include traumatic brain injury, stroke (particularly ischemic stroke), and all other neurological disorders associated with neuronal death including Parkinson's Disease and Huntington's Disease.

Therapeutic/prophylactic intervention to inhibit HSD1 expression and/or activity include but are not limited to administration of selective inhibitors shortly after a neurological injury event (e.g., a traumatic brain injury event or an ischemic episode), and chronic administration in individuals who have already suffered an injury event or are at higher risk for sufferering a neurological injury (e.g., stroke), and in genetically predisposed individuals.

Depending upon the individual's condition, the selective inhibitor can be administered in a therapeutic or prophylactic amount. If the individual has suffered a neurological injury, including hypoxia/ischemia, then for some period of time after the injury, the inhibitor is typically administered in a therapeutic amount. A “therapeutic amount,” as defined herein, means an amount sufficient to remedy a neurological disease state or symptoms, or otherwise prevent, hinder, retard or reverse the progression of a neurological disease or any other undesirable symptoms, especially stroke and more particularly ischemic stroke.

If an individual only presents with risk factors suggesting he or she is susceptible to neurological injury, then the agent is administered in a prophylactically effective amount. A prophylactic amount can also be administered as part of a long-term regimen for individuals that have already had a stroke and are at increased risk of another stroke. A “prophylactic amount” is an amount sufficient to prevent, hinder or retard a neurological disease or any undesirable symptom, particularly with regard to neurological disorders such as stroke, particularly ischemic stroke.

Prophylactic treatment can commence whenever an individual is at increased risk of suffering from a neurological disorder such as stroke. For example, individuals having risk factors known to be correlated with stroke can be administered prophylactic amounts of a selective HSD1 inhibitor.

The therapeutic agents of the present invention can also be administered in conjunction with other agents that are known to be useful to treat or ameliorate symptoms associated with neurological disorders or neuronal injuries. For example, administration of MgCl₂ has been shown to attenuate cortical histological damages following traumatic brain injury (Bareyre et al., J Neurotrauma 17: 1029-39, 2000). Antagonists of cholinergic or glutamatergic receptors (e.g., AMPA-glutamate receptor) may also be useful for alleviating symptoms of traumatic brain injury (see, Hamm et al, Cognitive Brain Research, 1, 223-226 (1993); and Lyeth et al, Brain Research, 452, 39-48 (1988)). Other agents useful for treating symptoms associated with TBI include, nefiracetam or its metabolites (see U.S. Pat. No. 6,348,489); bromocriptine (Petro et al., Arch Phys Med Rehabil 82:1637, 2001); bupropion (Teng et al., Brain Inj, 15: 463-7, 2001); high-dose human albumin (Ginsberg et al., J Neurosurg, 94: 499-509, 2001); and donepezil (Whelan et al., Ann Clin Psychiatry 12: 131-5, 2000). Additional useful agents have been described in, e.g., Hatton, CNS Drugs, 15: 553-81, 2001. Any of these agents can be administered together (concurrently or sequentially) with the therapeutic compositions of the present invention to treat a subject suffering from TBI.

Conventional methods of treatment for stroke often include thrombolytic agents, (see Deitcher and Jaff (2002) Rev Cardiovasc Med. 2002; 3 Suppl 2:S25-33. Thrombolytic agents include tissue plasminogen activator and derivatives thereof, e.g. monteplase, TNK-rt-PA, reteplase, lanoteplase, alteplase, pamiteplase. etc.; streptokinase; urokinase; APSAC; r-Prourokinase; heparin; staphylokinase; and the like. Any of these agents may be administered together (concurrently or sequentially) with the therapeutic compositions of the present invention to treat a subject following hypoxia/ischemia.

### Inhibitors of HSD1

Therapeutic agents for use in the methods of the invention are inhibitors of HSD1, preferably selective inhibitors of HSD1, as defined above, although in some instances non-selective inhibitor, e.g. carbenoxolone, may find use. In other embodiments, the inhibitor is a non-selective inhibitor other than carbenoxolone. Such selective and non-selective agents are known in the art.

Included as compounds of interest are the following compounds. Steroid inhibitors of 11β-HSD1, such as 11-keto testosterone, 11-keto-androsterone, etc. are disclosed in U.S.2003148987; and WO200241352. Triazole inhibitors of HSD1 are disclosed in WO0200365983; in WO200458730; in WO200489367; in WO200489380; in U.S. Pat. No. 6,730,690, in WO20040048912; in U.S.20040106664; in U.S.20040133011; WO2003104207; and in WO2003104208. 1,4-disubstituted piperidine inhibitors of HSD1 are disclosed in WO2004033427. 2-oxo-ethanesulfinamide derivative inhibitors of HSD1 are disclosed in WO2004011410; and WO200441264. Amide and substituted amide derivative inhibitors of HSD1 are disclosed in WO200465351, and in WO2004089470. Substituted pyrazolo[1,5-α]pyrimidine inhibitors of HSD1 are disclosed in WO2004089471. Other inhibitors are disclosed in WO2004027047; WO200456745; and WO200489896. Each of these references is herein specifically incorporated by reference for the teachings of compounds and formulations.

In one embodiment of the invention, the inhibitor has the general formula set forth in any one of WO03043999; WO03044009; WO03044000; WO0190091; WO0190090; WO0190094; including the generic structure, as defined therein:

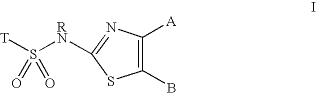

In another embodiment of the invention, the inhibitor has the general formula set forth in U.S. Pat. No. 6,730,690, including the generic structure as defined therein:

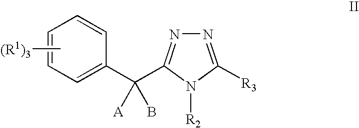

In onother embodiment of the invention, the HSD1 inhibitor has the general formula set forth in WO2004/02747, as defined therein: 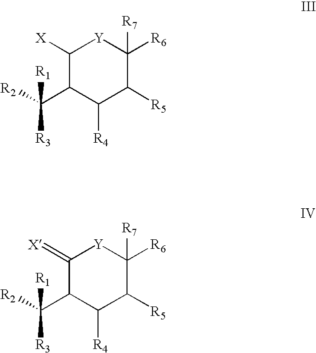
wherein R₁ is H or CH₃, R₂ is H, CH₃, or CH₂CH₃, R₃ is H, CH₃, CH₂CH₃ or CH₂CH₂CH₃, R₄ is H, CH₃, or CH₂CH₃, R₅ is H, CH₃, or CH₂CH₃, R₆ is H, CH₃, CH₂CH₃ or CH₂CH₂CH₃, R₇ is H or CH₃, X is OH, SH, or NH₂, X′ is O, S or NH, and Y is O, S, NH or CH₂.

Flavanones are another selective inhibitor for 11β-HSD1 (see Schweizer (2003) supra.) Included are substituted flavanones, particularly hydroxy derivatives, e.g. 2′-hydroxyflavanone; 4′-hydroxyflavanone; etc.

Inhibitory compounds may also be determined by screening compounds for effectiveness in inhibiting HSD1. Candidate compounds are preferably further screened for selectivity, and may be tested for in vivo efficacy. Such agents may include candidate drug compounds, genetic agents, e.g. coding sequences; ribozymes, catalytic RNAs, antisense compounds, polypeptides, e.g. factors, antibodies, etc.

Assays for determining inhibition of HSD1 are known in the art, for example as set forth in WO03/043999. HSD1 may be contacted with cortisone in the presence of suitable buffers and cofactors; and in the presence of candidate inhibitors. The ability of the enzyme to reduce the cortisone to cortisol is then assayed, e.g. by RIA, ELISA, etc. The selectivity of candidate inhibitors may be determined by performing a similar assay with HSD2 to verify that the compound substantially lacks inhibitory activity, as described above.

The neuroprotective activity of candidate compounds may be determined with in vitro and in vivo assays. For example, cell cultures are used in screening agents for their effect on neural and/or brain cells and neurologic events, e.g. during ischemia.

In one aspect, potential neuroprotective compounds are screened against oxygen and glucose deprivation (OGD) induced cell death in cell cultures. In general, removal of chemical energy to the neurons results in glutamate release thereby overactivating the receptors of the adjacent cells. The activated receptors are ionotropic ion channels, therefore, toxic concentration of calcium and sodium ions are achieved in the cells resulting in a delayed cell death after about 24 hours in culture. These conditions mimic ischemic stroke. OGD in cell cultures has been studied by exposing cultured tissue to media such as artificial cerebro-spinal fluid (aCSF), with an ion composition similar to that of the extracellular fluid of normal brain, with 2-6 mM K⁺, 1.5-3 mM Ca²⁺, 116 mM NaCl, 1 mM NaH₂PO₄, 26.2 mM NaHCO₃, 0.01 mM glycine in a glucose free media, and pH 7.4. The cells are maintained in the ischemic conditions for a period of time sufficient to induce a detectable effect, usually for at least about 90 min, preferably for at least about 60 minutes, and for not more than about 2 hours.

However, during ischemia the distribution of ions across cell membranes dramatically shifts. The co-pending and co-owned application U.S. Ser. No. 10/131,731 (herein incorporated by reference), provides a medium that more accurately reflects the extracellular fluid of the brain during an ischemic event. Thus, in another embodiment of a method for identifying ligands, the conditions and culture medium allow simulation of physiological and pathophysiological events affecting neural cells. Cultures of suitable cells or hippocampal slices are exposed transiently to a synthetic medium that reproduces the effects of ischemia. The cells or the slices are then monitored for the effect of the ischemic conditions on physiology, phenotype, etc.

In one aspect, the cells are an integrated system of brain tissue, with preserved synaptic connections and a diversity of cells including neurons, astrocytes and microglia. Such tissue can provide an in vitro model for pathophysiological events in the hippocampus following ischemia in vivo, including selective and delayed neuronal death in the CA1 region and increased damage by hyperglycemia.

Artificial ischemic cerebro-spinal fluid (iCSF), as used herein, refers to a glucose-free medium similar to the extracellular fluid of the brain during ischemia in vivo. The iCSF ionicity has a potassium concentration of at least about 50 mM, not more than about 90 mM, usually at least about 60 mM, not more than about 80 mM, and preferably about 65 to 75 mM K⁺, and in some instances about 70 mM K⁺. The concentration of calcium is at least about 0.1 mM, not more than about 1 mM, usually at least about 0.2 mM and not more than about 0.5 mM, preferably about 0.3 mM Ca²⁺. The pH of the iCSF media is at least about 6.7 and not more than about 6.9, preferably about pH 6.8.

The medium may be glucose free, or may comprise glucose at a concentration from about 10 mM to 100 mM, usually from about 25 mM to 75 mM, and may be about 40 mM. The cultures of the present invention show increased cell damage in the presence of glucose during ischemia, which simulates the in vivo effects of glucose. Hyperglycemia aggravates ischemic brain damage in vivo, and glucose in iCSF also significantly exacerbates cell damage following oxygen deprivation. This model of in vitro ischemia is useful in studies of the mechanisms and treatment of ischemic cell death.

The cells or hippocampal slices are maintained in the ischemic conditions for a period of time sufficient to induce a detectable effect, usually for at least about 5 minutes, more usually for at least about 1 minute, preferably for at least about 15 minutes, and for not more than about 1 hour. The non-hippocampal cells are maintained in the ischemic conditions for at least about 60 min, and not more than about 120 min preferably about 90 min.

Maintaining cultured cells or hippocampal slices in vitro in iCSF during oxygen glucose deprivation (OGD) provides a realistic simulation of in vivo events, which include a selective and delayed cell death in the CA1 region, assessed by propidium iodide uptake. Cell death is glutamate receptor dependent, as evidenced by the mitigation of damage by blockade of the N-methyl-D-aspartate and the α-Amino-3-hydroxy-5-methyl-4-isoxazolepropionic acid receptors.

Screening methods generally involve conducting various types of assays to identify agents that affect tissue damage that occurs during ischemia. Thus, a library of compounds is screened for potential neuroprotective compounds against oxygen-glucose deprivation (OGD) induced cell death in neuronal primary cultures. The library of compounds can be commercially available, can be proprietary, or can be custom synthesized. As fully explained above, when neurons are deprived of chemical energy, glutamate floods out of the neurons in which it is stored and over activates receptors in nearby cells. This leads to the entry of deadly amounts of calcium and sodium into the cells and causing a delayed cell death after 24 hours in culture. These conditions mimic the ischemic stroke.

Determining the in vivo efficacy of candidate compounds is also of particular interest. Candidate compounds may be administered to an animal in a model for stroke, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). One model for focal cerebral ischemia uses middle cerebral artery occlusion (MCAO) in rats. Studies in normotensive rats can produce a standardized and repeatable infarction. MCAO in the rat mimics the increase in plasma catecholamines, electrocardiographic changes, sympathetic nerve discharge, and myocytolysis seen in the human patient population.

### Formulations

Therapeutic agents, i.e. inhibitors of HSD1 as described above can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain. Intrathecal administration maybe carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. an NSAID such as indomethacin may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

A pharmaceutically or therapeutically effective amount of the composition is delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount may be in the range of about 0.001 mg/kg to about 100 mg/kg body weight, in at least one dose. The subject may be administered in as many doses as is required to reduce and/or alleviate the signs, symptoms, or causes of the disorder in question, or bring about any other desired alteration of a biological system.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

The following compound, also referred to as BVT 2733 (Barf et al J Med Chem, 45, 3813-3815), is a potent and selective murine HSD1 inhibitor with in vitro IC50=96 nM for mouse HSD1, with negligible affinity for human 11βHSD1 (IC 50=3341 nM) and inactive for human 11βHSD2 (IC 50>10 000 nM).

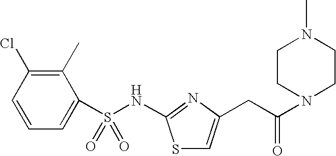

3-Chloro-2-methyl-N-{4-[2-(4-methyl-piperazin-1-yl)-2-oxo-ethyl]-thiazol-2-yl}-benzenesulfonamide

Alternatively, the following compound may also be used in testing:

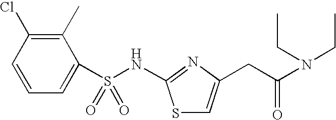

2-[2-(3-Chloro-2-methyl-benzenesulfonylamino)-thiazol-4-yl]-N,N-diethyl-acetamide

BVT 2733 was tested in the following MCAO protocol. A middle cerebral artery (MCA) occlusion was used to induce temporary cerebral ischemia. It involves anesthetizing the rat, making an incision in the ventral neck region to isolate the common carotid artery and the internal and external carotid arteries. The blood flow into the area is temporarily blocked by clamping off these arteries to allow the external carotid artery to be cut open. A silicone-coated mono filament is then inserted into the external carotid artery and woven through the artery into the internal carotid until it can occlude blood flow to the middle cerebral artery (MCA). The filament is removed after 90 min. After removal of the filament, the external carotid stump is tied shut and the clamps removed to allow the return of blood flow to the brain. The incision will be closed with wound clips. Post surgery, animals are observed until recovery from anesthesia.

During the surgery, animals are kept on a thermostat-regulated heating pad to maintain the body and head temperatures at 37° C. To inhibit the blood from clotting 90 IU/kg⁻¹ heparin is administered iv to animals after occlusion of MCAO.

The body core temperature of the animal is measured regularly, up to 1 day of recovery in some animals. Hypo or hyperthermia in animals is avoided by heating or cooling, respectively. Temperatures are measured manually using a rectal probe. The animals have access to both food and water during this period.

Animals were subjected to 90 minutes of transient focal ischemia by MCAO (middle cerebral artery occlusion) with an intraluminal filament technique (Toung et al., (1999) Stroke 30: 1279-1285). After reperfusion animals were treated with a bolus dose of BVT 2733 (90 mg/kg) or vehicle (5% DMSO in PEG) at 4.5 hours post Medial Cerebral Artery Occlusion followed by a second bolus dose (90 mg/kg) at 8.5 hours post-occlusion.

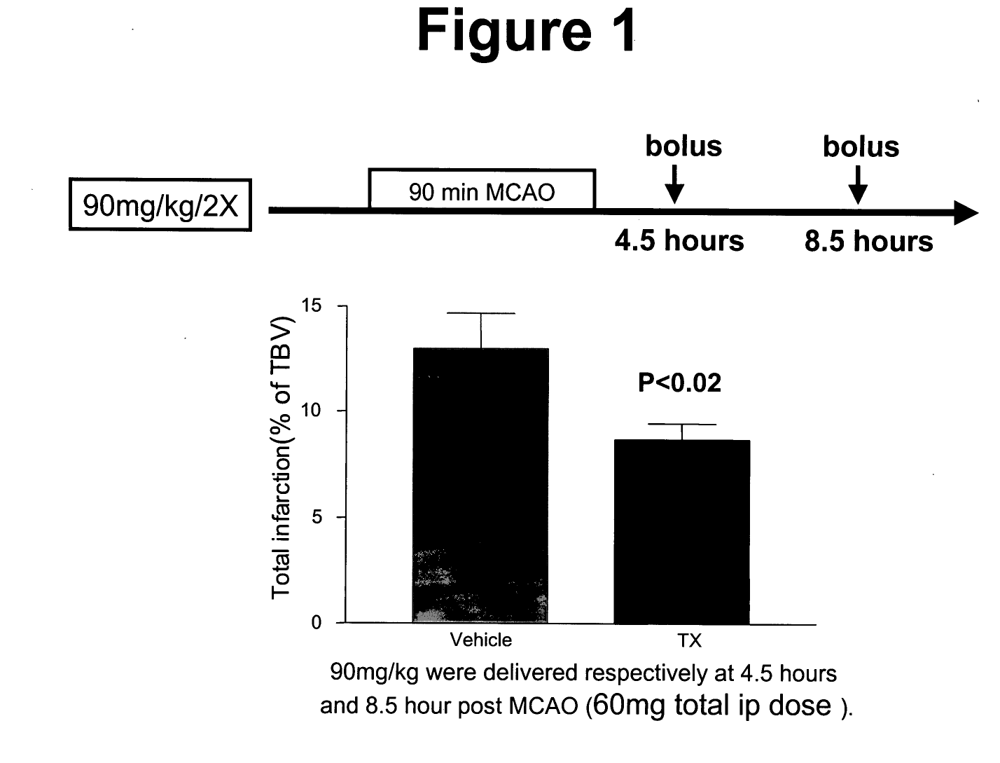

After 24 hours of reperfusion, the animals are sacrificed. The brains are harvested and sliced into coronal sections for staining with 1% triphenyltetrazolium chloride (TTC) in saline at 37° C. for 30 minutes. Infarction volume was measured by digital imaging and image analysis software. Infarction volumes are determined in cortex and striatum and expressed as a percentage of the total brain volume. The animals treated with BVT 2733 showed smaller infarction compared to vehicle treated rats. The neuroprotection observed was a potent and significant neuroprotection as depicted in FIG. 1. A more extended therapeutic window as well as lower doses of this compound are being explored.

### Example 2

### Animal Studies with Carbenoxolone

General method: The method used is a middle cerebral artery (MCA) occlusion to induce temporary cerebral ischemia. It involves anesthetizing the rat, making an incision in the ventral neck region to isolate the common carotid artery and the internal and external carotid arteries. The blood flow into the area is temporarily blocked by clamping off these arteries to allow the external carotid artery to be cut open. A silicone-coated mono filament is then inserted into the external carotid artery and woven through the artery into the internal carotid until it can occlude blood flow to the middle cerebral artery (MCA). The filament can then be tied in place (permanent occlusion) or removed after a short amount of time depending on the desired degree of ischemic damage (3 minutes to 2 hours). After removal of the filament, the external carotid stump is tied shut and the clamps removed to allow the return of blood flow to the brain. The incision was closed with wound clips. Post surgery, animals were observed until recovery from anesthesia.

During the surgery, animals were kept on a thermostat-regulated heating pad to maintain the body and head temperatures at 37° C. To inhibit the blood from clotting 90 IU.kg⁻¹ Heparin was administered intravenously to the animal after occlusion of MCAO. The body core temperature of the animal was measured regularly, up to 1 day of recovery in some animals. Hypo or hyperthermia in animals is avoided by heating or cooling, respectively. Temperatures are measured manually using a rat probe. The animals have access to both food and water during this period. These animals do not usually show any hyperthermia after 4 hours.

Animals were subjected to 90 minutes of transient focal ischemia by MCAO (middle cerebral artery occlusion) with an intraluminal filament technique (Toung et al. (1999) Stroke 30: 1279-1285). After reperfusion animals were treated with a bolus dose of carbenoxolone (20 mg) or vehicle (saline) at 5 min of reperfusion in addition to a continuous infusion (1.6 mg/h for 24 hours) or vehicle (vehicle; n=13; CBX=11). Moreover, a potent neuroprotection was observed when carbenoxolone was administered at 4 to 6 hours post-occlusion.

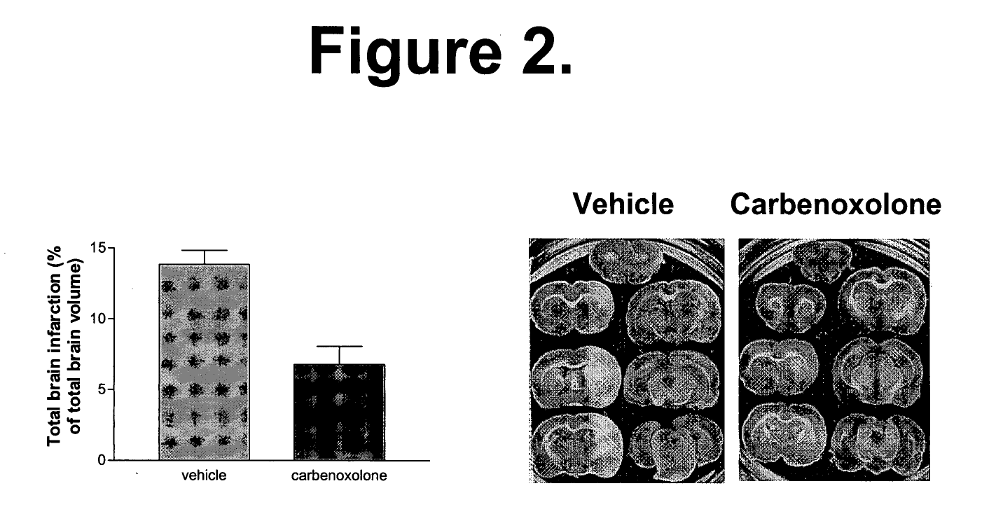

After 24 hours of reperfusion, the animals were sacrificed. The brains are harvested and sliced into coronal sections for staining with 1% triphenyltetrazolium chloride (TTC) in saline at 37° C. for 30 minutes. Infarction volume was measured by digital imaging and image analysis software. Infarction volumes are determined in cortex and striatum an expressed as a percentage of the total brain volume the animal treated with carbenoxolone showed smaller infarction compared to vehicle treated rats. The compositions thus provide neuroprotection to the animals (FIG. 2).

1. A method for treating or preventing a neurologic disorder associated with neuronal death in a subject animal, the method comprising:
administering to said subject an effective amount of an inhibitor of 11 β hydroxysteroid dehydrogenase 1 (HSD1).

2. The method of claim 1, wherein said neurologic disorder results from exposure of neurons to hypoxia/ischemia.

3. The method of claim 2, wherein said hypoxia/ischemia is caused by stroke, cardiac arrest or perinatal asphyxia.

4. The method of claim 3, wherein said administering is performed after a stroke.

5. The method of claim 4, wherein the administering is performed within about 24 hours after said stroke.

6. The method of claim 5, wherein the administering is performed within about 3 hours after said stroke.

7. The method of claim 5, wherein the administering is performed within about 6 hours after said stroke.

8. The method of claim 1, wherein said inhibitor is a selective inhibitor of HSD1.

9. The method of claim 8, wherein said inhibitor is: 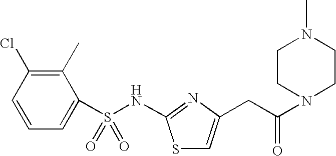
3-Chloro-2-methyl-N-{4-[2-(4-methyl-piperazin-1-yl)-2-oxo-ethyl]-thiazol-2-yl}-benzenesulfonamide

10. The method of claim 8, wherein said inhibitor is 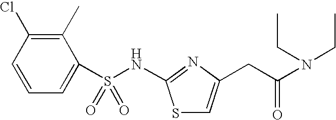
2-[2-(3-Chloro-2-methyl-benzenesulfonylamino)-thiazol-4-yl]-N,N-diethyl-acetamide

11. The method according to claim 1, wherein said neurologic disorder results from traumatic brain injury.

12. The method according to claim 1, wherein said neurologic disorder is Parkinson's Disease.

13. A method of screening a candidate compound for efficacy in preventing adverse effects of a neurologic disorder associated with neuronal death, the method comprising:
assaying said compound for inhibition of HSD1 to determine an HSD1 inhibitor;
contacting a model for said neurologic disorder with said HSD1 inhibitor; and
determining the efficacy of said compound in preventing said adverse effects.

14. The method according to claim 13, further comprising the step of determining the selectivity of said candidate compound for inhibition of HSD1 and not HSD2.

15. The method according to claim 13, wherein said model for said neurologic disorder with said HSD1 inhibitor is an in vitro model.

16. The method according to claim 15, wherein said model comprises oxygen and glucose deprivation (OGD) induced cell death in cell cultures.

17. The method according to claim 15, wherein said model comprises cultures of suitable cells or hippocampal slices are exposed transiently to a synthetic medium that reproduces the effects of ischemia.

18. The method according to claim 15, wherein said model comprises an animal model.

19. The method according to claim 15, wherein said model comprises a middle cerebral artery occlusion (MCAO) in rats.
